(19) United States
(12) Patent Application Publication
(10) Pub. No.: US 2026/0137884 A1
(43) Pub. Date: May 21, 2026

(54) SYSTEMS, APPARATUSES, AND METHODS FOR DISLODGING MUCUS IN THE LUNGS OF A PATIENT

(71) Applicant: TRIAD NATIONAL SECURITY, LLC, Los Alamos, NM (US)

(72) Inventors: Cristian PANTEA, Los Alamos, NM (US); John James GREENHALL, Los Alamos, NM (US)

(21) Appl. No.: 19/371,562

(22) Filed: Oct. 28, 2025

Related U.S. Application Data
(60) Provisional application No. 63/722,949, filed on Nov. 20, 2024.

(51) Int. Cl. A61M 16/00; A61B 5/00; A61B 34/10

(52) U.S. Cl. CPC A61M 16/0006; A61B 5/0036; A61B 5/004; A61B 34/10; A61B 2034/105

(57) ABSTRACT

Systems, apparatuses, and methods for dislodging mucus in the lungs of a patient are disclosed herein. For example, an embodiment in this disclosure includes a system that includes an acoustic device. In some embodiments, the system includes an apparatus comprising memory and one or more processors communicatively coupled to the memory. In some embodiments, the one or more processors are configured to receive patient data associated with a patient. In some embodiments, the one or more processors are configured to determine, using the lungs model, a resonant frequency of the affected area of the lungs. In some embodiments, the one or more processors are configured to cause the acoustic device to generate one or more acoustic signals. In some embodiments, the one or more acoustic signals are configured to cause vibration of the affected area of the lungs at the resonant frequency.

### CROSS-REFERENCE TO RELATED APPLICATIONS

This application claims the benefit of U.S. Provisional Application No. 63/722,949, filed Nov. 20, 2024, the entire contents of which are incorporated by reference herein.

### STATEMENT REGARDING GOVERNMENT FUNDING

The United States government has rights in this invention pursuant to Contract Number 89233218CNA000001 between the U.S. Department of Energy and Triad National Security, LLC for operation of Los Alamos National Laboratory. The government has certain rights in the invention.

### BACKGROUND

Applicant has identified many challenges and difficulties associated with dislodging mucus in the lungs of a patient. Through applied effort, ingenuity, and innovation, Applicant has solved problems related to dislodging mucus in the lungs of a patient by developing solutions embodied in the present disclosure, which are described in detail below.

### SUMMARY

Various embodiments are generally directed towards dislodging mucus in the lungs of a patient.

In accordance with one aspect of the disclosure, a system is provided. In some embodiments, the system includes an acoustic device. In some embodiments, the system includes an apparatus comprising memory and one or more processors communicatively coupled to the memory. In some embodiments, the one or more processors are configured to receive patient data associated with a patient. In some embodiments, the one or more processors are configured to generate a lungs model based on the patient data. In some embodiments, the lungs model is representative of lungs of the patient. In some embodiments, the lungs of the patient include an affected area. In some embodiments, the affected area of the lungs comprises mucus. In some embodiments, the one or more processors are configured to determine, using the lungs model, a resonant frequency of the affected area of the lungs. In some embodiments, the one or more processors are configured to cause the acoustic device to generate one or more acoustic signals. In some embodiments, the one or more acoustic signals are configured to cause vibration of the affected area of the lungs at the resonant frequency.

In some embodiments, the patient data is representative of one or more of an age of the patient, a height of the patient, a weight of the patient, or a condition associated with the patient.

In some embodiments, the patient data is representative of one or more images generated by a computed tomography (CT) scan or a magnetic resonance imaging (MRI) scan.

In some embodiments, the acoustic device is an invasive device configured to be at least partially inserted into the patient.

In some embodiments, the acoustic device is at least partially inserted into the patient when the patient is ventilated.

In some embodiments, the acoustic device is a non-invasive device.

In some embodiments, the acoustic device comprises one or more of an acoustic transducer, an air-coupled acoustic transducer, a direct-coupled transducer, a speaker, or a shaker.

In some embodiments, to determine the resonant frequency for the affected area comprises the one or more processors being configured to process the lungs model using a finite element modeling technique.

In some embodiments, the resonant frequency is determined based on a location of the affected area in the lungs.

In some embodiments, the resonant frequency is less when the affected area is located in a first section of the lungs than when the affected area is located in a second section of the lungs.

In some embodiments, the first section is associated with a first size and the second section is associated with a second size.

In some embodiments, the first size is greater than the second size.

In some embodiments, the vibration of the affected area of the lungs causes the mucus in the affected area to be dislodged from the affected area.

In some embodiments, the system includes a mechanical device configured to provide one or more air pulses to the lungs of the patient.

In accordance with another aspect of the disclosure a method is provided. In some embodiments, the method includes receiving patient data associated with a patient. In some embodiments, the method includes generating a lungs model based on the patient data. In some embodiments, the lungs model is representative of lungs of the patient. In some embodiments, the lungs of the patient include an affected area. In some embodiments, the affected area of the lungs comprises mucus. In some embodiments, the method includes determining, using the lungs model, a resonant frequency of the affected area of the lungs. In some embodiments, the method includes causing the acoustic device to generate one or more acoustic signals. In some embodiments, the one or more acoustic signals are configured to cause vibration of the affected area of the lungs at the resonant frequency.

In some embodiments, to determine the resonant frequency for the affected area comprises processing the lungs model using a finite element modeling technique.

In some embodiments, the patient data is representative of one or more of an age of the patient, a height of the patient, a weight of the patient, or a condition associated with the patient.

In some embodiments, the patient data is representative of one or more images generated by a computed tomography (CT) scan or a magnetic resonance imaging (MRI) scan.

In some embodiments, the resonant frequency is determined based on a location of the affected area in the lungs.

In some embodiments, the method includes causing a mechanical device to provide one or more air pulses to the lungs of the patient.

In accordance with another aspect of the disclosure an apparatus is provided. In some embodiments, the apparatus includes memory and one or more processors communicatively coupled to the memory. In some embodiments, the one or more processors are configured to receive patient data associated with a patient. In some embodiments, the one or more processors are configured to generate a lungs model based on the patient data. In some embodiments, the lungs model is representative of lungs of the patient. In some embodiments, the lungs of the patient include an affected area. In some embodiments, the affected area of the lungs comprises mucus. In some embodiments, the one or more processors are configured to determine, using the lungs model, a resonant frequency of the affected area of the lungs. In some embodiments, the one or more processors are configured to cause the acoustic device to generate one or more acoustic signals. In some embodiments, the one or more acoustic signals are configured to cause vibration of the affected area of the lungs at the resonant frequency.

The above summary is provided merely for purposes of summarizing some example embodiments to provide a basic understanding of some aspects of the invention. Accordingly, it will be appreciated that the above-described embodiments are merely examples and should not be construed to narrow the scope or spirit of the invention in any way. It will be appreciated that the scope of the invention encompasses many potential embodiments in addition to those here summarized, some of which will be further described below.

### BRIEF DESCRIPTION OF THE DRAWINGS

Having thus described certain example embodiments of the present disclosure in general terms above, non-limiting and non-exhaustive embodiments of the subject disclosure will now be described with reference to the accompanying drawings which are not necessarily drawn to scale. The components illustrated in the accompanying drawings may or may not be present in certain embodiments described herein. Some embodiments may include fewer (or more) components than those shown in the drawings. Some embodiments may include the components arranged in a different way:

FIG. 1 illustrates an environment in which one or more embodiments of the present disclosure may operate;

FIG. 2 illustrates a schematic view of lungs of a patient in accordance with one or more embodiments of the present disclosure;

FIG. 3 illustrates a schematic view of a patient in accordance with one or more embodiments of the present disclosure;

FIG. 4 illustrates a flowchart of an example method in accordance with one or more embodiments of the present disclosure; and

FIG. 5 illustrates an example computer processing device in accordance with one or more embodiments of the present disclosure.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Further, “based on,” “based at least in part on,” “based at least on,” “based upon,” and/or similar words are used herein interchangeably in an open-ended manner such that they do not indicate being based only on or based solely on the referenced element or elements unless so indicated. Like numbers refer to like elements throughout.

### Overview

Embodiments of the present disclosure are directed to systems, apparatuses, and methods for dislodging mucus in the lungs of a patient. In some examples, systems, apparatuses, and methods for dislodging mucus in the lungs of a patient are desirable for generating a lungs model representative of the lungs of a patient. In some embodiments, systems, apparatuses, and methods for dislodging mucus in the lungs of a patient are desirable for dislodging mucus in the lungs of a patient. In this way, conditions that are affecting a patient, such as cystic fibrosis, pneumonia, chronic obstructive pulmonary disease (COPD), and/or the like, may be identified and/or treated.

Example solutions for dislodging mucus in the lungs of a patient including providing pulses of air into a patient's airway. However, such example solutions are inaccurate, ineffective, and inefficient. For example, such example solutions are inaccurate because such example solutions do not include generating a lungs model representative of the lungs of a patient. As a result, such example solutions are unable to obtain an understanding of the conditions affecting the patient (e.g., where the mucus is in the lungs) and/or obtain an accurate representation of the patient's lungs (e.g., the size of specific portions of the lungs). As another example, such example solutions are ineffective because such example solutions are limited to using pulses of air to dislodge mucus. As a result, such example solutions are not able to precisely target specific areas of the lungs to dislodge mucus. As another example, such example solutions are inefficient because such example solutions require a patient to be ventilated in order to operate. Accordingly, there is a need for systems, apparatuses, and methods that are able to dislodge mucus in the lungs of a patient in an accurate, effective, and efficient manner.

Thus, to address these and/or other issues related to dislodging mucus in the lungs of a patient, systems, apparatuses, and methods for dislodging mucus in the lungs of a patient are disclosed herein. For example, an embodiment, in this disclosure, described in greater detail below, includes a system that includes an acoustic device. In some embodiments, the system includes an apparatus comprising memory and one or more processors communicatively coupled to the memory. In some embodiments, the one or more processors are configured to receive patient data associated with a patient. In some embodiments, the one or more processors are configured to generate a lungs model based on the patient data. In some embodiments, the lungs model is representative of lungs of the patient. In some embodiments, the lungs of the patient include an affected area. In some embodiments, the affected area of the lungs comprises mucus. In some embodiments, the one or more processors are configured to determine, using the lungs model, a resonant frequency of the affected area of the lungs. In some embodiments, the one or more processors are configured to cause the acoustic device to generate one or more acoustic signals. In some embodiments, the one or more acoustic signals are configured to cause vibration of the affected area of the lungs at the resonant frequency. Accordingly, the systems, apparatuses, and methods disclosed herein enable dislodging mucus in the lungs of a patient in an accurate, effective, and efficient manner.

### Example Systems and Apparatuses

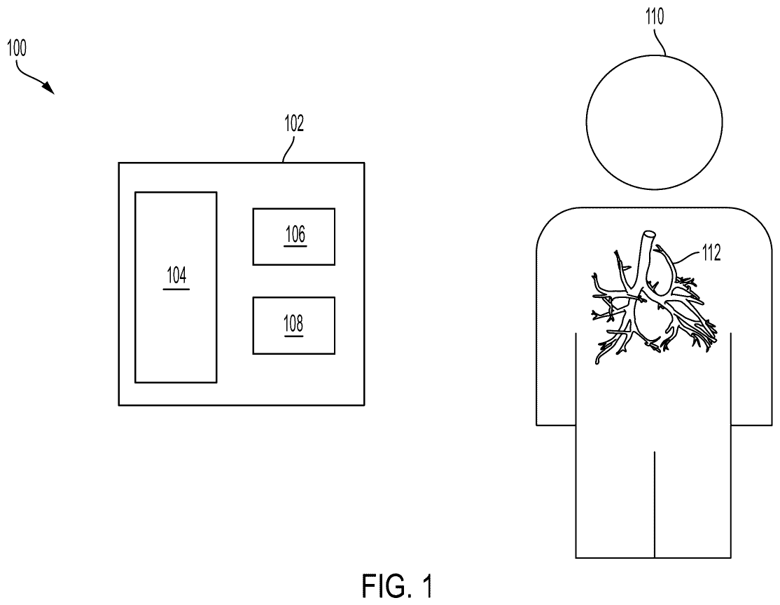

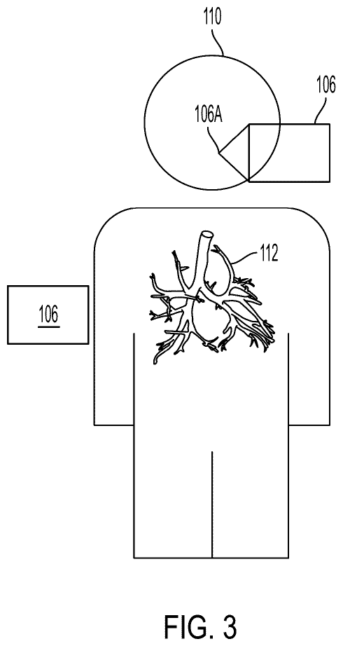

With reference to FIGS. 1-3, an environment 100 in which embodiments of the present disclosure may operate is illustrated. In some embodiments, the environment 100 is associated with a clinical setting. In this regard, for example, the environment 100 may be one or more of a hospital, a medical clinic, a medical practice, and emergency department, an urgent care center, and/or the like.

In some embodiments, the environment 100 includes a patient 110. In some embodiments, the patient 110 is an individual who is associated with environment 100. In this regard, in some embodiments, the patient 110 may be an individual who is receiving medical treatment for a condition associated with the patient 110. In some embodiments, a condition associated with the patient 110 includes a medical condition that is affecting the patient 110. For example, a condition associated with the patient 110 may include cystic fibrosis, pneumonia, chronic obstructive pulmonary disease (COPD), and/or the like.

In some embodiments, the patient 110 includes lungs 112. In some embodiments, the lungs 112 include an affected area 118. In some embodiments, the affected area 118 includes mucus. In this regard, in some embodiments, the affected area 118 is an area of the lungs 112 that has a buildup of mucus that is desirable to dislodge. For example, it may be desirable to dislodge mucus in the affected area 118 in order to treat and/or mitigate a condition associated with the patient 110.

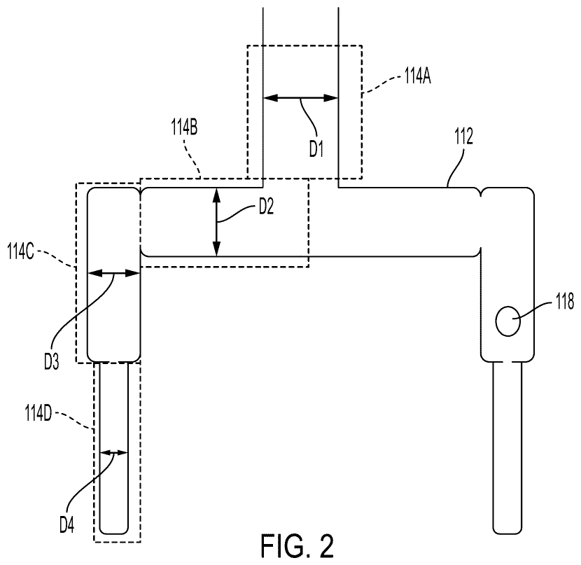

In some embodiments, the lungs 112 include a plurality of sections 114. For example, as depicted in FIG. 2, the plurality of sections may include a first section 114A, a second section 114B, a third section 114C, and/or a fourth section 114D. In some embodiments, each of the plurality of sections 114 is associated with one or more dimensions. For example, the first section 114A may be associated with one or more first dimensions (D1), the second section 114B may be associated with one or more second dimensions (D2), the third section 114C may be associated with one or more third dimensions (D3), and/or the fourth section 114D may be associated with one or more fourth dimensions (D4).

In some embodiments, the one or more dimensions associated with a section of the plurality of sections 114 is indicative of a size of the section. Additionally, or alternatively, the one or more dimensions associated with a section of the plurality of sections 114 is indicative of a volume of the section. Additionally, or alternatively, the one or more dimensions associated with a section of the plurality of sections 114 is indicative of an area of the section. Additionally, or alternatively, the one or more dimensions associated with a section of the plurality of sections 114 is indicative of a shape of the section. In this regard, for example, the one or more first dimensions (D1) may be indicative of a size, volume, area, shape, and/or the like of the first section 114A. As another example, the one or more second dimensions (D2) may be indicative of a size, volume, area, shape, and/or the like of the second section 114B. As another example, the one or more third dimensions (D3) may be indicative of a size, volume, area, shape, and/or the like of the third section 114C. As another example, the one or more fourth dimensions (D4) may be indicative of a size, volume, area, shape, and/or the like of the fourth section 114D.

In some embodiments, the one or more dimensions of each of the plurality of sections 114 are different. In this regard, in some embodiments, the size, volume, area, shape, and/or the like of each of the plurality of sections 114 may be different. For example, the size, volume, area, shape, and/or the like of the first section 114A may be different than the size, volume, area, shape, and/or the like of the second section 114B. In this regard, for example, the first section 114A may have a greater volume than the second section 114B. As another example, the first section 114A may have a greater size than the second section 114B. Although the lungs 112 are depicted in FIG. 2 as having four sections in the plurality of sections 114, it would be understood by one skilled in the field to which this disclosure pertains that the plurality of sections 114 may include more or less than four sections.

In some embodiments, the environment 100 includes a system 102. In some embodiments, the system 102 includes an apparatus 104. Additionally, or alternatively, the system 102 includes an acoustic device 106. Additionally, or alternatively, the system 102 includes a mechanical device 108. Although the apparatus 104, the acoustic device 106, and the mechanical device 108 are depicted as separate devices in FIG. 1, it would be understood by one skilled in the field to which this disclosure pertains that the apparatus 104, the acoustic device 106, and the mechanical device 108 could be combined into a single device.

In some embodiments, the apparatus 104 is configured to receive patient data. In some embodiments, the patient data is associated with the patient 110. In some embodiments, patient data includes one or more items of data representative and/or indicative of one or more characteristics of the patient 110. For example, patient data may include one or more items of data representative and/or indicative of an age of the patient 110. As another example, patient data may include one or more items of data representative and/or indicative of a height of the patient 110. As another example, patient data may include one or more items of data representative and/or indicative of a weight of the patient 110. As another example, patient data may include one or more items of data representative and/or indicative of a condition associated with the patient 110 (e.g., cystic fibrosis).

Additionally, or alternatively, patient data includes one or more items of data representative and/or indicative of one or more images associated with the patient 110. For example, patient data may include one or more items of data representative and/or indicative of one or more images generated by a computed tomography (CT) scan of the patient 110. As another example, patient data may include one or more items of data representative and/or indicative of one or more images generated by a magnetic resonance imaging (MRI) scan of the patient 110.

In some embodiments, the apparatus 104 is configured to receive patient data. In some embodiments, the apparatus 104 may be configured to receive patient data from a CT machine that has generated one or more images associated with the patient by performing a CT scan of the patient 110. In some embodiments, the apparatus 104 may be configured to receive patient data from an MRI machine that has generated one or more images associated with the patient by performing an MRI scan of the patient 110. In some embodiments, the apparatus 104 may be configured to receive patient data from the patient 110 and/or a medical professional associated with the patient 110 (e.g., a medical professional providing treatment to the patient 110). For example, the patient 110 and/or a medical professional may be able to provide patient data to the apparatus 104 via an interface. In some embodiments, the apparatus 104 is configured to generate patient data. For example, the apparatus 104 may be configured to receive a first portion of patient data and use the first portion of patient data to generate a second portion (e.g., use one or more images associated with the patient 110 to determine a condition associated with the patient 110).

In some embodiments, the apparatus 104 is configured to generate a lungs model. In some embodiments, the lungs model is a computing model that is representative of the lungs 112 of the patient 110. For example, the lungs model may be a 3-dimensional computing model representative of the lungs 112. In this regard, in some embodiments, the lungs model is representative and/or indicative of the plurality of sections 114 of the lungs 112 and/or the dimensions of each of the plurality of sections 114. For example, the lungs model may be representative of the one or more first dimensions (D1), the one or more second dimensions (D2), the one or more third dimensions (D3), and/or the one or more fourth dimensions (D4). Additionally, or alternatively, the lungs model is representative and/or indicative of a condition associated with the patient 110. For example, the lungs model is representative and/or indicative of cystic fibrosis, pneumonia, chronic obstructive pulmonary disease (COPD), and/or the like associated with the patient 110. Said differently, for example, the lungs model may be representative and/or indicative of the impact a condition associated with the patient 110 is having on the lungs 112 of the patient 110.

In some embodiments, the apparatus 104 is configured to generate the lungs model based on patient data associated with the patient 110. In this regard, for example, the apparatus 104 may be configured to generate the lungs model using patient data representative of one or more images generated by a CT scan and/or an MRI scan of the lungs 112 of the patient 110. As another example, the apparatus 104 may be configured to generate the lungs model using patient data representative of an age, weight, height, and/or condition of the patient 110 (e.g., the dimensions of the lungs 112 indicated by the lungs model is determined based on the age or height of the patient 110).

In some embodiments, the apparatus 104 is configured to determine a resonant frequency for the affected area 118 of the lungs 112. In some embodiments, the apparatus 104 is configured to determine a resonant frequency for the affected area 118 based on a location of the affected area 118 in the lungs 112. In this regard, in some embodiments, the dimensions of the location of the affected area 118 in the lungs 112 may impact the resonant frequency of the affected area 118. For example, if the affected area 118 is located in the first section 114A having one or more first dimensions (D1) the apparatus 104 may be configured to determine a first resonant frequency for the affected area 118. As another example, if the affected area 118 is located in the second section 114B having one or more second dimensions (D2) the apparatus 104 may be configured to determine a second resonant frequency for the affected area 118. As another example, if the affected area 118 is located in the third section 114C having one or more third dimensions (D3) the apparatus 104 may be configured to determine a third resonant frequency for the affected area 118. As another example, if the affected area 118 is located in the fourth section 114D having one or more fourth dimensions (D4) the apparatus 104 may be configured to determine a fourth resonant frequency for the affected area 118. Said differently, for example, the apparatus 104 may be configured to determine a resonant frequency for the affected area 118 based on the size, volume, area, and/or shape of the location in which the affected area 118 is located in the lungs 112.

In some embodiments, the apparatus 104 is configured to determine the resonant frequency for the affected area 118 of the lungs 112 using the lungs model. In this regard, in some embodiments, the apparatus 104 is configured to determine a resonant frequency for the affected area 118 by processing the lungs model using a finite element modeling technique. For example, the apparatus 104 may be configured to use a finite element modeling technique to determine the resonant frequency for the affected area 118 based on the location of the affected area 118 in the lungs 112 (e.g., the dimensions associated with the affected area 118).

In some embodiments, the greater the size of a section of the plurality of sections 114 of the lungs 112, the lower the resonant frequency of the affected area 118. In this regard for example, if the affected area 118 is located in the first section 114A and the first section is greater in size that the second section 114B, the resonant frequency associated with the affected area 118 is less than if the affected area 118 was located in the second section 114B. Additionally, or alternatively, the greater the volume of a section of the plurality of sections 114 of the lungs 112, the lower the resonant frequency of the affected area 118. In this regard for example, if the affected area 118 is located in the first section 114A and the first section is greater in volume that the second section 114B, the resonant frequency associated with the affected area 118 is less than if the affected area 118 was located in the second section 114B.

In some embodiments, the apparatus 104 is configured to cause the acoustic device 106 to generate one or more acoustic signals. In some embodiments, the one or more acoustic signals are configured to cause vibration of the affected area 118 of the lungs 112 at the resonant frequency of the affected area 118. In this regard, by causing vibration of the affected area 118 of the lungs 112 at the resonant frequency of the affected area 118, mucus in the affected area 118 may be dislodged and/or broken apart.

In some embodiments, the acoustic device 106 is one or more of an acoustic transducer, an air-coupled acoustic transducer, a direct-coupled transducer, a speaker, a shaker, and/or the like. In some embodiments, the acoustic device 106 is a non-invasive device. In this regard, when the acoustic device 106 is a non-invasive device, the acoustic device 106 is configured to be positioned proximate the patient 110 in order to provide one or more acoustic signals to the affected area 118 of the lungs 112. For example, when the acoustic device 106 is a non-invasive device, the acoustic device 106 is configured to be positioned next to the chest of the patient 110 such that the acoustic signals are able to cause the affected area 118 to vibrate at the resonant frequency. As another example, when the acoustic device 106 is a non-invasive device, the acoustic device 106 is configured to be positioned such that it is in contact with the patient 110 (e.g., in contact with the chest of the patient 110) such that the acoustic signals are able to cause the affected area 118 to vibrate at the resonant frequency.

In some embodiments, the acoustic device 106 is an invasive device. In this regard, when the acoustic device 106 is an invasive device, at least a first portion 106A of the acoustic device 106 is configured to be at least partially inserted into the patient 110. For example, the first portion 106A of the acoustic device 106 may be inserted into the mouth or nose of the patient 110. In this regard, when the acoustic device 106 is an invasive device, the acoustic device 106 is configured to provide one or more acoustic signals to the affected area 118 via an airway of the patient 110. In some embodiments, the acoustic device 106 is at least partially inserted into the patient 110 when the patient 110 is ventilated. Said differently, for example, when the patient 110 is ventilated the acoustic device 106 may be an invasive device and when the patient 110 is not ventilated the acoustic device 106 may be a non-invasive device.

In some embodiments, the mechanical device 108 is configured to provide one or more air pulses to the lungs 112 of the patient 110. In some embodiments, the one or more air pulses are configured to dislodge mucus in the affected area 118. In this regard, in some embodiments, the system 102 is configured to use one or more of air pulses and/or acoustic signals to dislodge mucus from the affected area 118.

In some embodiments, the apparatus 104 is configured to cause the mechanical device 108 to provide one or more air pulses to the lungs 112. In some embodiments, the apparatus 104 is configured to cause the mechanical device 108 to provide one or more air pulses to the lungs 112 at the same time as causing the apparatus 104 to generate one or more acoustic signals. Additionally, or alternatively, the apparatus 104 is configured to cause the mechanical device 108 to provide one or more air pulses to the lungs 112 after causing the apparatus 104 to generate one or more acoustic signals. Additionally, or alternatively, the apparatus 104 is configured to cause the mechanical device 108 to provide one or more air pulses to the lungs 112 before causing the apparatus 104 to generate one or more acoustic signals. In this regard, for example, the system 102 may be configured to alternate between air pulses and acoustic signals for dislodging mucus in the affected area 118.

### Example Methods

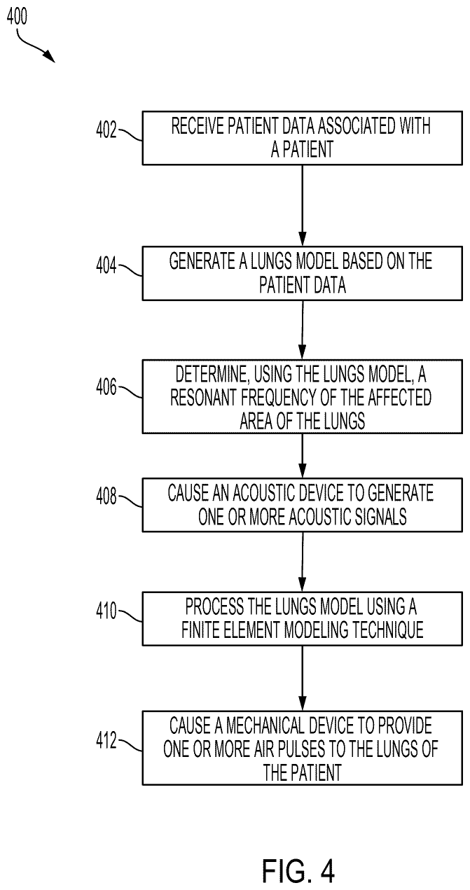

FIG. 4 is a flowchart broadly illustrating an example method 400 that is performed to perform dislodging mucus in the lungs of a patient in accordance with one or more embodiments of the present disclosure. In some embodiments, the method 400 is performed using one or more components of the system 102. For example, the method 400 may be performed using the system 102, the apparatus 104, the acoustic device 106, and/or the mechanical device 108.

As shown in block 402, the method 400 may include receiving patient data associated with a patient. As described above, in some embodiments,, patient data includes one or more items of data representative and/or indicative of one or more characteristics of the patient. For example, patient data may include one or more items of data representative and/or indicative of an age of the patient. As another example, patient data may include one or more items of data representative and/or indicative of a height of the patient. As another example, patient data may include one or more items of data representative and/or indicative of a weight of the patient. As another example, patient data may include one or more items of data representative and/or indicative of a condition associated with the patient (e.g., cystic fibrosis).

Additionally, or alternatively, patient data includes one or more items of data representative and/or indicative of one or more images associated with the patient. For example, patient data may include one or more items of data representative and/or indicative of one or more images generated by a computed tomography (CT) scan of the patient. As another example, patient data may include one or more items of data representative and/or indicative of one or more images generated by a magnetic resonance imaging (MRI) scan of the patient.

In some embodiments, the apparatus is configured to receive patient data. In some embodiments, the apparatus may be configured to receive patient data from a CT machine that has generated one or more images associated with the patient by performing a CT scan of the patient. In some embodiments, the apparatus may be configured to receive patient data from an MRI machine that has generated one or more images associated with the patient by performing an MRI scan of the patient. In some embodiments, the apparatus may be configured to receive patient data from the patient and/or a medical professional associated with the patient (e.g., a medical professional providing treatment to the patient). For example, the patient and/or a medical professional may be able to provide patient data to the apparatus via an interface. In some embodiments, the apparatus is configured to generate patient data. For example, the apparatus may be configured to receive a first portion of patient data and use the first portion of patient data to generate a second portion (e.g., use one or more images associated with the patient to determine a condition associated with the patient).

As shown in block 404, the method 400 may include generating a lungs model based on the patient data. As described above, in some embodiments, the lungs model is a computing model that is representative of the lungs of the patient. For example, the lungs model may be a 3-dimensional computing model representative of the lungs. In this regard, in some embodiments, the lungs model is representative and/or indicative of the plurality of sections of the lungs and/or the dimensions of each of the plurality of sections. For example, the lungs model may be representative of the one or more first dimensions (D1), the one or more second dimensions (D2), the one or more third dimensions (D3), and/or the one or more fourth dimensions (D4). Additionally, or alternatively, the lungs model is representative and/or indicative of a condition associated with the patient. For example, the lungs model is representative and/or indicative of cystic fibrosis, pneumonia, chronic obstructive pulmonary disease (COPD), and/or the like associated with the patient. Said differently, for example, the lungs model may be representative and/or indicative of the impact a condition associated with the patient is having on the lungs of the patient.

As shown in block 406, the method 400 may include determining, using the lungs model, a resonant frequency of the affected area of the lungs. As described above, in some embodiments, the apparatus is configured to determine a resonant frequency for the affected area based on a location of the affected area in the lungs. In this regard, in some embodiments, the dimensions of the location of the affected area in the lungs may impact the resonant frequency of the affected area. For example, if the affected area is located in the first section having one or more first dimensions (D1) the apparatus may be configured to determine a first resonant frequency for the affected area. As another example, if the affected area is located in the second section having one or more second dimensions (D2) the apparatus may be configured to determine a second resonant frequency for the affected area. As another example, if the affected area is located in the third section having one or more third dimensions (D3) the apparatus may be configured to determine a third resonant frequency for the affected area. As another example, if the affected area is located in the fourth section having one or more fourth dimensions (D4) the apparatus may be configured to determine a fourth resonant frequency for the affected area. Said differently, for example, the apparatus may be configured to determine a resonant frequency for the affected area based on the size, volume, area, and/or shape of the location in which the affected area is located in the lungs.

As shown in block 408, the method 400 may include causing an acoustic device to generate one or more acoustic signals. As described above, in some embodiments, the one or more acoustic signals are configured to cause vibration of the affected area of the lungs at the resonant frequency of the affected area. In this regard, by causing vibration of the affected area of the lungs at the resonant frequency of the affected area, mucus in the affected area may be dislodged and/or broken apart.

As shown in block 410, the method 400 may include processing the lungs model using a finite element modeling technique. As described above, in some embodiments, the apparatus is configured to determine a resonant frequency for the affected area by processing the lungs model using a finite element modeling technique. For example, the apparatus may be configured to use a finite element modeling technique to determine the resonant frequency for the affected area based on the location of the affected area in the lungs (e.g., the dimensions associated with the affected area).

As shown in block 412, the method 400 may include causing a mechanical device to provide one or more air pulses to the lungs of the patient. As described above, in some embodiments, the mechanical device is configured to provide one or more air pulses to the lungs of the patient. In some embodiments, the one or more air pulses are configured to dislodge mucus in the affected area. In this regard, in some embodiments, the system is configured to use one or more of air pulses and/or acoustic signals to dislodge mucus from the affected area.

In some embodiments, the apparatus is configured to cause the mechanical device to provide one or more air pulses to the lungs. In some embodiments, the apparatus is configured to cause the mechanical device to provide one or more air pulses to the lungs at the same time as causing the apparatus to generate one or more acoustic signals. Additionally, or alternatively, the apparatus is configured to cause the mechanical device to provide one or more air pulses to the lungs after causing the apparatus to generate one or more acoustic signals. Additionally, or alternatively, the apparatus is configured to cause the mechanical device to provide one or more air pulses to the lungs before causing the apparatus to generate one or more acoustic signals. In this regard, for example, the system may be configured to alternate between air pulses and acoustic signals for dislodging mucus in the affected area.

### Example Computer Processing Device

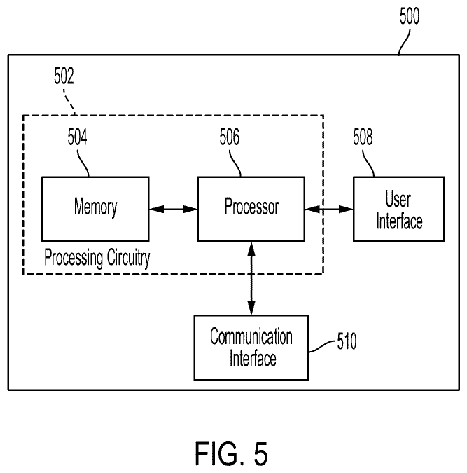

With reference to FIG. 5, a block diagram of an example computer processing device 500 is illustrated in accordance with some example embodiments. In some embodiments, the system 102, the apparatus 104, the acoustic device 106, and/or the mechanical device 108 may be embodied as one or more computer processing devices, such as the computer processing device 500 in FIG. 5. However, it should be noted that the components, devices, or elements illustrated in and described with respect to FIG. 5 below may not be mandatory and thus one or more may be omitted in certain embodiments. Additionally, some embodiments may include further or different components, devices or elements beyond those illustrated in and described with respect to FIG. 5.

The computer processing device 500 may include or otherwise be in communication with processing circuitry 502 that is configurable to perform actions in accordance with one or more embodiments disclosed herein. In this regard, the processing circuitry 502 may be configured to perform and/or control performance of one or more functionalities of the computer processing device 500 in accordance with various embodiments, and thus may provide means for performing functionalities of the computer processing device 500 in accordance with various embodiments. The processing circuitry 502 may be configured to perform data processing, application execution and/or other processing and management services according to one or more embodiments. In some embodiments, the computer processing device 500 or a portion(s) or component(s) thereof, such as the processing circuitry 502, may be embodied as or comprise a chip or chip set. In other words, the computer processing device 500 or the processing circuitry 502 may comprise one or more physical packages (e.g., chips) including materials, components and/or wires on a structural assembly (e.g., a baseboard). The structural assembly may provide physical strength, conservation of size, and/or limitation of electrical interaction for component circuitry included thereon. The computer processing device 500 or the processing circuitry 502 may therefore, in some cases, be configured to implement an embodiment of the disclosure on a single chip or as a single “system on a chip.” As such, in some cases, a chip or chipset may constitute means for performing one or more operations for providing the functionalities described herein.

In some embodiments, the processing circuitry 502 may include a processor 506 and, in some embodiments, such as that illustrated in FIG. 5, may further include memory 504. The processing circuitry 502 may be in communication with or otherwise control a user interface 508 and/or a communication interface 510. As such, the processing circuitry 502 may be embodied as a circuit chip (e.g., an integrated circuit chip) configured (e.g., with hardware, software or a combination of hardware and software) to perform operations described herein.

The processor 506 may be embodied in a number of different ways. For example, the processor 506 may be embodied as various processing means such as one or more of a microprocessor or other processing element, a coprocessor, a controller or various other computing or processing devices including integrated circuits such as, for example, an ASIC (application specific integrated circuit), an FPGA (field programmable gate array), or the like. Although illustrated as a single processor, it will be appreciated that the processor 506 may comprise a plurality of processors. The plurality of processors may be in operative communication with each other and may be collectively configured to perform one or more functionalities of the computer processing device 500 as described herein. In some embodiments, the processor 506 may be configured to execute instructions stored in the memory 504 or otherwise accessible to the processor 506. As such, whether configured by hardware or by a combination of hardware and software, the processor 506 may represent an entity (e.g., physically embodied in circuitry—in the form of processing circuitry 502) capable of performing operations according to embodiments of the present disclosure while configured accordingly. Thus, for example, when the processor 506 is embodied as an ASIC, FPGA or the like, the processor 506 may be specifically configured hardware for conducting the operations described herein. Alternatively, as another example, when the processor 506 is embodied as an executor of software instructions, the instructions may specifically configure the processor 506 to perform one or more operations described herein.

In some embodiments, the memory 504 may include one or more non-transitory memory devices such as, for example, volatile and/or non-volatile memory that may be either fixed or removable. In this regard, the memory 504 may comprise a non-transitory computer-readable storage medium. It will be appreciated that while the memory 504 is illustrated as a single memory, the memory 504 may comprise a plurality of memories. The memory 504 may be configured to store information, data, applications, instructions and/or the like for enabling the computer processing device 500 to carry out various functions in accordance with one or more embodiments. For example, the memory 504 may be configured to buffer input data for processing by the processor 506. Additionally, or alternatively, the memory 504 may be configured to store instructions for execution by the processor 506. As yet another alternative, the memory 504 may include one or more databases that may store a variety of files, contents or data sets. Among the contents of the memory 504, applications may be stored for execution by the processor 506 in order to carry out the functionality associated with each respective application. In some cases, the memory 504 may be in communication with one or more of the processor 506, user interface 508, and/or communication interface 510 via a bus(es) for passing information among components of the computer processing device 500.

The user interface 508 may be in communication with the processing circuitry 502 to receive an indication of a user input at the user interface 508 and/or to provide an audible, visual, mechanical or other output to the user. As such, the user interface 508 may include, for example, a keyboard, a mouse, a joystick, a display, a touch screen display, a microphone, a speaker, and/or other input/output mechanisms. As such, the user interface 508 may, in some embodiments, provide means for a user to access and interact with the system 102, the apparatus 104, the acoustic device 106, and/or the mechanical device 108.

The communication interface 510 may include one or more interface mechanisms for enabling communication with other devices and/or networks. In some cases, the communication interface 510 may be any means such as a device or circuitry embodied in either hardware, or a combination of hardware and software that is configured to receive and/or transmit data from/to a network and/or any other device or module in communication with the processing circuitry 502. By way of example, the communication interface 510 may be configured to enable communication between the system 102, the apparatus 104, the acoustic device 106, the mechanical device 108 and/or other devices. Accordingly, the communication interface 510 may, for example, include an antenna (or multiple antennas) and supporting hardware and/or software for enabling communications with a wireless communication network (e.g., a wireless local area network, cellular network, global positing system network, and/or the like) and/or a communication modem or other hardware/software for supporting communication via cable, digital subscriber line (DSL), universal serial bus (USB), Ethernet or other methods.

### Conclusion

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

1. A system comprising:
an acoustic device; and
an apparatus comprising memory and one or more processors communicatively coupled to the memory, the one or more processors configured to:
receive patient data associated with a patient;
generate a lungs model based on the patient data, wherein the lungs model is representative of lungs of the patient, wherein the lungs of the patient include an affected area, wherein the affected area of the lungs comprises mucus;
determine, using the lungs model, a resonant frequency of the affected area of the lungs; and
cause the acoustic device to generate one or more acoustic signals, wherein the one or more acoustic signals are configured to cause vibration of the affected area of the lungs at the resonant frequency.

2. The system of claim 1, wherein the patient data is representative of one or more of an age of the patient, a height of the patient, a weight of the patient, or a condition associated with the patient.

3. The system of claim 1, wherein the patient data is representative of one or more images generated by a computed tomography (CT) scan or a magnetic resonance imaging (MRI) scan.

4. The system of claim 1, wherein the acoustic device is an invasive device configured to be at least partially inserted into the patient.

5. The system of claim 4, wherein the acoustic device is at least partially inserted into the patient when the patient is ventilated.

6. The system of claim 1, wherein the acoustic device is a non-invasive device.

7. The system of claim 1, wherein the acoustic device comprises one or more of an acoustic transducer, an air-coupled acoustic transducer, a direct-coupled transducer, a speaker, or a shaker.

8. The system of claim 1, wherein to determine the resonant frequency for the affected area comprises the one or more processors being configured to:
process the lungs model using a finite element modeling technique.

9. The system of claim 1, wherein the resonant frequency is determined based on a location of the affected area in the lungs.

10. The system of claim 9, wherein the resonant frequency is less when the affected area is located in a first section of the lungs than when the affected area is located in a second section of the lungs.

11. The system of claim 10, wherein the first section is associated with a first size and the second section is associated with a second size, wherein the first size is greater than the second size.

12. The system of claim 1, wherein the vibration of the affected area of the lungs causes the mucus in the affected area to be dislodged from the affected area.

13. The system of claim 1, further comprising:
a mechanical device configured to provide one or more air pulses to the lungs of the patient.

14. A method comprising:
receiving patient data associated with a patient;
generating a lungs model based on the patient data, wherein the lungs model is representative of lungs of the patient, wherein the lungs of the patient include an affected area, wherein the affected area of the lungs comprises mucus;
determining, using the lungs model, a resonant frequency of the affected area of the lungs; and
causing an acoustic device to generate one or more acoustic signals, wherein the one or more acoustic signals are configured to cause vibration of the affected area of the lungs at the resonant frequency.

15. The method of claim 14, wherein the patient data is representative of one or more of an age of the patient, a height of the patient, a weight of the patient, or a condition associated with the patient.

16. The method of claim 14, wherein the patient data is representative of one or more images generated by a computed tomography (CT) scan or a magnetic resonance imaging (MRI) scan.

17. The method of claim 14, wherein to determine the resonant frequency for the affected area comprises:
processing the lungs model using a finite element modeling technique.

18. The method of claim 14, wherein the resonant frequency is determined based on a location of the affected area in the lungs.

19. The method of claim 14, further comprising:
causing a mechanical device to provide one or more air pulses to the lungs of the patient.

20. An apparatus comprising memory and one or more processors communicatively coupled to the memory, the one or more processors configured to:
receive patient data associated with a patient;
generate a lungs model based on the patient data, wherein the lungs model is representative of lungs of the patient, wherein the lungs of the patient include an affected area, wherein the affected area of the lungs comprises mucus;
determine, using the lungs model, a resonant frequency of the affected area of the lungs; and
cause an acoustic device to generate one or more acoustic signals, wherein the one or more acoustic signals are configured to cause vibration of the affected area of the lungs at the resonant frequency.
